(19) United States
(12) Patent Application Publication
(10) Pub. No.: US 2020/0170965 A1
(43) Pub. Date: Jun. 4, 2020

(54) METHODS OF TREATING DOOSE SYNDROME USING FENFLURAMINE

(71) Applicant: ZOGENIX INTERNATIONAL LIMITED, Berkshire (GB)

(72) Inventors: Brooks BOYD, Berkeley, CA (US); Stephen J. FARR, Orinda, CA (US); Bradley GALER, West Chester, PA (US)

(73) Assignee: ZOGENIX INTERNATIONAL LIMITED, Berkshire (GB)

(21) Appl. No.: 16/611,610

(22) PCT Filed: May 4, 2018
(86) PCT No.: PCT/GB2018/051210

§ 371 Date: Nov. 7, 2019

Related U.S. Application Data
(60) Provisional application No. 62/503,638, filed on May 9, 2017.
(60) Provisional application No. 62/581,375, filed on Nov. 3, 2017.

(51) Int. Cl. A61K 31/135; A61P 25/08; A61K 9/70; A61K 45/06; A61K 9/00

(52) U.S. Cl. CPC A61K 31/135; A61P 25/08; A61K 9/0053; A61K 45/06; A61K 9/7023

(57) ABSTRACT

A method of treating and/or preventing symptoms of Doose syndrome in a patient such as a patient previously diagnosed with Doose syndrome, by administering an effective dose of fenfluramine or its pharmaceutically acceptable salt to that patient. Doose syndrome patients are treated at a preferred dose of less than about 10.0 to about 0.01 mg/kg/day.

### FIELD OF THE INVENTION

This invention relates generally to the field of methods of treatment and in particular, methods of treating human patients, and more particularly to methods and compositions useful in treating human patients diagnosed with Doose Syndrome.

### BACKGROUND OF THE INVENTION

This invention relates to the treatment of symptoms of Doose Syndrome in patients diagnosed with Doose syndrome using an amphetamine derivative, specifically fenfluramine.

Epilepsy is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited disorders or degenerative disease, head injury or trauma, metabolic disorders, cerebrovascular accident and alcohol withdrawal.

A large number of epilepsy subtypes have been characterized and systematically categorized according to their own unique clinical symptoms, signs, and phenotype, underlying pathophysiology and distinct responses to different treatments. The most recent version of this categorization scheme, and the one that is widely accepted in the art, is that adopted by the International League Against Epilepsy's (“ILAE”) Commission on Classification and Terminology as shown in Table 1 below. See Berg et al., “Revised terminology and concepts for organization of seizures,” Epilepsia, 51(4):676-685 (2010).

TABLE 1
ILAE Classification Scheme for Epilepsy Subtypes
I. ELECTROCHEMICAL SYNDROMES (by age of onset)

| | |
|---|---|
| A. Neonatal period | 1. Benign familial neonatal epilepsy (BFNE) |
| | 2. Early myoclonic encephalopathy (EME) |
| | 3. Ohtahara syndrome |
| B. Infancy | 1. Epilepsy of infancy with migrating focal seizures |
| | 2. West syndrome |
| | 3. Myoclonic epilepsy in infancy (MEI) |
| | 4. Benign infantile epilepsy |
| | 5. Benign familial infantile epilepsy |
| | 6. Dravet syndrome |
| | 7. Myoclonic encephalopathy in non-progressive disorders |
| C. Childhood | 1. Febrile seizures plus (FS+) (can start in infancy) |
| | 2. Panayiotopoulos syndrome |
| | 3. Epilepsy with myoclonic atonic (previously astatic) seizures |
| | (Doose syndrome) |
| | 4. Benign epilepsy with centrotemporal spikes (BECTS) |
| | 5. Autosomal-dominant nocturnal frontal lobe epilepsy (ADNFLE) |
| | 6. Late onset childhood occipital epilepsy (Gastaut type) |
| | 7. Epilepsy with myoclonic absences |
| | 8. Lennox-Gastaut syndrome |
| | 9. Epileptic encephalopathy with continuous spike-and-wave during |
| | 10. Landau-Kleffner syndrome (LKS) |
| | 11. Childhood absence epilepsy (CAE) |
| D. Adolescence-Adult | 1. Familial focal epilepsy with variable foci (childhood to adult) |
| | 2. Reflex epilepsies |
| E. Less specific age relationship | 1. Familial focal epilepsy with variable foci (childhood to adult) |
| | 2. Reflex epilepsies |

II. DISTINCTIVE CONSTELLATIONS

| | |
|---|---|
| A. Mesial temporal lobe epilepsy with | |
| hippocampal sclerosis (MTLE with HS) | |
| B. Rasmussen syndrome | |
| C. Gelastic seizures with hypothalamic | |
| hamartoma | |
| D. Hemiconvulsion-hemiplegia-epilepsy | |
| E. Epilepsies that do not fit into any of these | 1. Presumed cause (presence or absence of a known structural or |
| diagnostic categories, | metabolic condition) |
| | 2. Primary mode of seizure onset (generalized vs. focal) |

III. EPILEPSIES ATTRIBUTED TO AND ORGANIZED BY STRUCTURAL-METABOLIC CAUSES

A. Malformations of cortical development
(hemimegalencephaly, heterotopias, etc.,)
B. Neurocutaneous syndromes (tuberous
sclerosis complex, Sturge-Weber, etc.,)
C. Tumor
D. Infection
E. Trauma

IV. ANGIOMA

A. Perinatal insults
B. Stroke
C. Other causes

V. EPILEPSIES OF UNKNOWN CAUSE
VI. CONDITIONS WITH EPILEPTIC SEIZURES NOT TRADITIONALLY DIAGNOSED AS FORMS
OF EPILEPSY PER SE

A. Benign neonatal seizures (BNS)
B. Febrile seizures (FS)

Those skilled in the art will recognize that different subtypes of epilepsy are triggered by different stimuli, are controlled by different biological pathways, and have different causes, whether genetic, environmental, and/or due to disease or injury of the brain. Further, inclusion of Part V of the ILAE classification scheme underscores the fact that a comprehensive understanding of epilepsy is still evolving, and that there are still subtypes of epilepsy that have not yet been fully characterized, or that remain unrecognized as distinct syndromes. Thus, the skilled artisan will recognize that teachings relating to one epileptic subtype are most commonly not necessarily applicable to any other subtype.

A large number of compounds, representing a variety of mechanisms of action, are used to treat different types of epilepsy. Table 2 below, although not comprehensive, includes those drugs which are most widely prescribed:

TABLE 2
Commonly Prescribed Anti-epileptic Drugs

| Generic Name | Trade Names |
|---|---|
| carbamazepine | Carbatrol, Epitol, Equetro, Tegretol |
| Clobazam | Frisium, Onfi |
| Clonazepam | Klonopin |
| Diazepam | Diastat, Valium |
| Ezogabine | Potiga |
| eslicarbazepine acetate | Aptiom |
| Ethosuximide | Zarontin |
| Felbamate | Felbatol |
| Fosphenytoin | Cerebyx |
| Gabapentin | Gralise, Horizant, Neurontin, Gabarone |
| Lacosamide | Vimpat |
| Lamotrigine | LaMICtal |
| Levetiracetam | Elepsia, Keppra, Levetiractam Stavzor |
| Lorazepam | Ativan |
| oxcarbazepine | Trileptal, Oxtellar |
| Perampanel | Fycompa |
| phenobarbital | Luminal, Solfoton |
| Phenytoin | Dilantin, Prompt, Di-Phen, Epanutin, Phenytek |
| Pregabalin | Lyrica |
| Primidone | Mysoline |
| Rufinamide | Banzel, Inovelon |
| Tiagabine | Gabitril |
| Topiramate | Qudexy XR, Topamax, Topiragen, Trokendi XR, |
| valproate, valproic | Depacon, Depakene, Depakote, |
| acid | |
| Vigabatrin | Sabril |
| Zonisamide | Zonegran |

Given the heterogeneity among epilepsy syndromes, it is not surprising that different epilepsy subtypes respond differently to different anticonvulsant drugs. That is, while a particular drug can be effective against one form of epilepsy, it can be wholly ineffective against others, or even contra-indicated due to exacerbation of symptoms, such as worsening the frequency and severity of the seizures. It follows that the efficacy of a particular drug with respect to a particular type of epilepsy is unpredictable, and therefore the discovery that a particular drug is effective in treating in treating a type of epilepsy for which that drug was not previously known to be effective is nearly always surprising, even in cases where the drug is known to be effective against other epilepsy types. This is especially true for drugs which are found to be effective in treating syndromes which were unresponsive to existing treatments.

### Doose Syndrome

### Background

An overview of Doose syndrome is provided in a recent review article. See Kelley et al., Developmental Medicine and Child Neurology (2010), p 989, DOI: 10.1111/j.1469-8749.2010.03744.

Doose syndrome is a form of refractory epilepsy for which few treatment options currently exist. It is relatively uncommon, with an incidence of about 1 in 10 000 children, constituting approximately 1 to 2% of childhood-onset epilepsies. It was first described as an independent epilepsy by Dr. Hermann Doose in 1970, and is currently categorized as “epilepsy with myoclonic-atonic seizures” or “myoclonic-astatic epilepsy” (see I(C)(3) in Table 1 above).

The diagnostic criteria for diagnosing Doose syndrome is based on the description of the seizures—myoclonic-astatic seizures, which is exclusive to MAE and is one of the defining characteristics of this syndrome. In addition, other features of this condition include: (1) absence of organic or other obvious cause for seizures; (2) onset of myoclonic-astatic seizures between 7 months and 6 years of age; (3) male:female ratio of 2:1 (1:1 in first year of life); (4) frequently, a hereditary predisposition; (5) varied seizure types, including myoclonic, astatic, myoclonic-astatic, absence, tonic, clonic, generalized tonic-clonic; (6) status epilepticus is common; and (7) EEG that is initially normal (or shows background theta), but subsequently shows generalized polyspike and wave epileptiform activity; and (8) clinical aspects that are not consistent with Dravet syndrome, Lennox-Gastaut syndrome, or benign myoclonic epilepsy.

Clinical presentation of Doose syndrome varies. Initial onset occurs within the first 5 years of life in 94% of cases, usually between 3 and 4 years of age, with 24% of children experiencing their first seizure in the first year of life. Some children may present with frequent explosive-onset seizures, in others further seizures may not occur for some time. In patients who experience their first seizure after 4 years of age, the initial manifestation is more likely to be absence seizures.

A number of clinical features are common among patients suffering from Doose syndrome. They develop normally until onset of seizures. The syndrome is associated with multiple different seizure types, including myoclonic seizures, and may be severe or more moderate. All seizure types can result in status epilepticus, including non-convulsive status, as well as myoclonic and absence status epilepticus.

The EEG of a Doose patient may be initially normal, but with progression of the disease will demonstrate a variety of abnormalities. Most commonly, the abnormal EEG will demonstrate frequent synchronous (generalized) spike wave activity at times in brief bursts of 2 to 5 Hz. However, despite the observed abnormalities, the overall posterior background rhythms and sleep architecture of children is generally normal. While Doose syndrome is thought to be a generalized seizure disorder, it is possible to see pseudofoci of activity on the EEG, which may shift in laterality. In younger individuals, the EEG may show continuous irregular activity which looks similar to hypsarrhythmia. During status epilepticus, rhythms consisting of continuous spike wave activity with interposed slow waves can be seen, which can lead to clinically unpredictable myoclonus occurring in multiple parts of the individual's body. Ibid.

Persons skilled in the art of diagnosing and treating patients recognize that, while Doose exhibits some clinical similarities to both Dravet syndrome and to Lennox-Gastaut syndrome in its early stages, Doose syndrome is a distinct medical condition with different underlying etiologies, symptomatology, EEG findings. Most importantly, Doose patients' responses to therapeutic interventions, especially their responses to pharmaceutical medications, mean in many cases that drugs which are effective for other forms of refractory epilepsy are not effective, or are strongly contraindicated, when treating Doose patients.

### Etiology

Since its first description in 1970, knowledge of Doose syndrome has continued to grow, but an understanding of Doose syndrome is still evolving. Its exact etiology is currently unknown.

It is currently believed that genetics plays an important role in the disease, with approximately 1 out of 3 family members of children with Doose Syndrome experiencing seizures. Doose was the first to point out the high incidence of both seizures and similar EEG findings among the family members of affected individuals (See Doose H et al., Centrencephalic myoclonic-astatic petit mal. Clinical and genetic investigations. Neuropediatrie 1970; 2: 59-78. (In German)). The prevalence of abnormal EEG findings was found to be 68% among immediate family members and up to 80% if distant relatives were included. Although the prevalence of specifically myoclonic and astatic seizures among family members was found to be only about 2%, this is 200 times higher than in the general population. Doose H., Myoclonic-astatic epilepsy, Epilepsy Res 1992; 6 (Suppl.) 163-8. Early papers reported that clinical seizures occurred in 35 to 40% of relatives of individuals with Doose syndrome. Id; See also Oguni H et al., Treatment and long-term prognosis of myoclonic-astatic epilepsy of early childhood,” Neuropediatrics 2002; 33: 122-32. Photosensitivity and abnormal theta background rhythm are the most common EEG findings. The likelihood of multifactorial inheritance is partly demonstrated by the heterogeneity in seizure manifestations among Doose patients. CHD2 (15q26), GABRG2 (5q34), SCN1A (2q24.3), SCN1B (19q13.12), SLC2A1 (1p34.2), and SLC6A1 (3p25.3) all appear to be implicated in Doose syndrome irrespective of any family history of GEFS+ disorder.

Doose patients were among the first among patients with GEFS+ disorder to be diagnosed with SCN1A mutations. A point mutation in exon 20 of SCN1A was discovered in a family in which one brother has severe myoclonic epilepsy and one has Doose syndrome, probably inherited from a father who had one febrile seizure and a few generalized tonic-clonic seizures throughout his life. Scheffer I., Generalized epilepsy with febrile seizures plus. A genetic disorder with heterogeneous clinical phenotypes. Brain 1997; 120: 479-90.

Individuals with Doose syndrome have also been found to have sodium channel subunit beta-1 (SCN1B) and gamma-aminobutyric acid receptor subunit gamma-2 (GABRG2) mutations, however they have not been found consistently in sporadic cases, suggesting that these gene mutations are unlikely to be the primary cause of Doose syndrome.

Mutations in the SLC2A1 gene (1p34.2) may also be implicated in a large number of patients: overall, up to 10% of patients with MAE have been reported to carry non-causal SLC2A1 mutations. And recently, some patients with MAE-like phenotypes were found to have mutations in CHD2 (15q26). (http://www.orpha.net/consor/cgi-bin/OC_Exp.php?Lng=GB&Expert=1942)

Even more recently, a large group (644 patients) with epileptic encephalopathies were screened for mutations in SLC6A1 (3p25.3), a gene initially identified in a large exome study. The results of the study were as follows: (1) SLC6A1 mutations, most of them de novo mutations, were found in a total of 7 patients; (2) all 7 patients who possessed an SLC6A1 mutation also exhibited an MAE phenotype; and (3) the 7 MAE patients who possessed an SLC6A1 represented almost 5% of the total number of patients with MAE. These findings, taken together, strongly suggest that the SLC6A1 gene plays some causative role in Doose syndrome. See Carville et al., “Mutations in the GABA Transporter SLC6A1 Cause Epilepsy with Myoclonic-Atonic Seizures,” Am J Hum Genet. 2015 May 7; 96(5):808-15. doi: 10.1016/j.ajhg.2015.02.016. Epub 2015 Apr. 9. Notably, 2 of the 7 mutations were truncating mutations, suggesting that the disease mechanism is haploinsufficiency

SLC6A1, also called GAT-1, is a transporter that removes GABA from the synaptic cleft. GABA is the main inhibitory transmitter in the brain, Without being bound by theory, SLC6A1 mutations that reduce the functional amount of GAT-1 on the pre-synapse should increase both the duration and the quantity of GABA in the synaptic cleft. However, how the supposed increase in GABA leads to epilepsy is entirely unclear.

Aside from genetics, there have been some reports of children with Doose syndrome who have identified underlying structural abnormalities, and thus symptomatic-structural etiologies to explain the phenotype. However, genetics and structural abnormalities cannot explain the Doose syndrome phenotype in all patients, and the majority of myoclonic-astatic epilepsy (MAE) cases remain to be explained.

### Prognosis and Treatment

The prognosis for Doose varies, and outcomes can range from normal cognition to severe intellectual disability, and from spontaneous remission to intractability. In approximately ⅔rds of Doose patients, the seizures resolve over time. Disease outcomes are not usually predictable in the first year of disease, although disease progression (resulting in episodes of status epilepticus, including tonic vibratory seizures and myoclonic status) as well as cognitive decline reflect unfavorable prognosis.

Treatment of Doose syndrome has historically been challenging, and the optimum treatment for Doose syndrome has yet to be established. A variety of treatment options exist or are being investigated, including pharmaceutical agents, ketogenic diet, and vagus nerve stimulation. However, there are drawbacks to each; further, few are reliably effective in the majority of patients, and none prevent seizures entirely. Thus, the process of identifying a treatment regimen that is effective for individual patients remains largely empirical.

Ketogenic diet is the most widely reported therapy for Doose syndrome, and may be the most efficacious. However, it is generally used as a second- or third-line treatment after one or two anticonvulsants have been tried, and has not been studied as a first-line treatment. Vagal nerve stimulation is another potential treatment option; however, to date there has been only a single reported case of its use, and it neither prevented or reduced seizures in the patient who used it.

As mentioned above, of the conventional antiepileptic drugs currently in use, many are ineffective or contraindicated for Doose syndrome patients (for example, carbamazepine, phenytoin, oxcarbazepine and vigabatrin). Some show inconsistent effects, reducing seizures in some patients but worsening them in others. A handful of anticonvulsants have proven useful for some patients but certainly not for most with Doose syndrome. Ethosuximide may be effective for patients, particularly where absence seizures are the primary seizure type. Valproic acid, and lamotrigine are also useful treatment options, and appear to have synergistic effects for some patients when given together. However, a recent case study reports valproate triggering status epilepticus in an 8-year-old male patient. See Grande-Martin et al., “Tonic Seizure Status Epilepticus Triggered by Valproate in a Child with Doose Syndrome” in Neuropediatrics. 2016 June; 47(3):187-9. doi: 10.1055/s-0036-1579632. Epub 2016 Mar. 15. Lamotrigine can also be problematic, because it can cause paradoxical worsening in some patients; moreover, dosage must be titrated slowly to prevent Stevens-Johnson syndrome (a form of toxic epidermal necrolysis which may progress to full-blown TEN resulting in detachment of more than 30% of body surface area.). Anecdotal use of levetiracetam and zonisamide may has shown some efficacy. Information on the use of clobazam and the newer anticonvulsants (e.g., rufinamide and lacosamide) is currently unavailable.

Thus overall, most Doose patients do not respond with significant seizure reduction to their polypharmacy regimen, but continue to have refractory and debilitating seizures while being treated with several anti-epileptic drugs concurrently. There is therefore a need for pharmaceutical agents which will be reliably effective in significantly decreasing or eliminating seizures in the majority of Doose syndrome patients without intolerable side effects.

### Fenfluramine

One drug that has been shown to be effective in other types of epilepsy is fenfluramine.

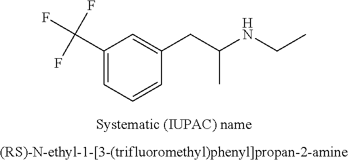 [SMILES: CCNC(C)Cc1cccc(C(F)(F)F)c1]

Fenfluramine is metabolized in vivo into norfenfluramine by cytochrome P450 enzymes in the liver. Such metabolism includes cleavage of an N-ethyl group to produce norfenfluramine as shown below.

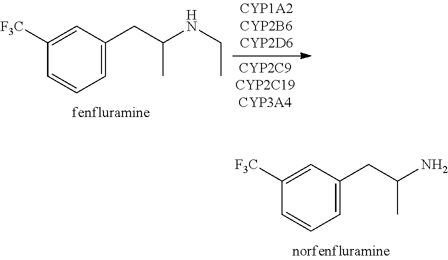 [SMILES: CCNC(C)Cc1cccc(C(F)(F)F)c1 ; CC(N)Cc1cccc(C(F)(F)F)c1]

Fenfluramine was first marketed in the US in 1973 and was administered in combination with phentermine to prevent and treat obesity. However, in 1997, it was withdrawn from the US and global market as its use was associated with the onset of cardiac valve fibrosis and pulmonary hypertension. Subsequently, the drug was withdrawn from sale globally and is no longer indicated for use in any therapeutic area. It is now thought that fenfluramine's toxicity is primarily associated with its primary metabolite norfenfluramine. Norfenfluramine is an agonist of the 5-HT2B receptor, which is associated with heart valve hypertrophy.

Initial investigations of fenfluramine as an anti-epileptic shared a common paradigm, i.e., that fenfluramine's primary effects were to inhibit behaviors that caused or induced seizures, vs. to prevent or ameliorate seizure activity. More recently, fenfluramine has been shown to be effective in treating Dravet syndrome and Lennox-Gastaut syndrome, both forms of refractory pediatric onset epilepsy. While these disorders arise in infancy and childhood, their underlying causes are unknown and the particulars of why fenfluramine is effective in each case are unclear. Moreover, as discussed above, there is a great deal of uncertainty with respect to the efficacy of anti-epileptics, and just because a drug is effective against one epileptic condition or one type of seizure cannot predict its potential efficacy for another.

In contrast, the literature is silent on the use of fenfluramine as a treatment for Doose syndrome, nor have experts in the field proposed such a use. This is unsurprising given that the underlying cause of Doose is as yet unknown and is a completely unique and different epilepsy condition than others; and the fact that many of the conventional anti-seizure medications that have been tried are ineffective, exacerbate symptoms, or show paradoxical effects among individuals.

### Problem Presented

The foregoing discussion makes clear that Doose is a serious illness which, if left untreated, can result in permanent cognitive impairment and even death. Current treatment options are limited to a small handful of pharmaceuticals and ketogenic diet, which for most patients are unsatisfactory. However, identifying new and novel effective treatment regimens for individual patients is largely empirical; further, there are significant drawbacks to each. In the case of pharmaceutical agents, efficacy is unpredictable and often incomplete, certain agents can worsen symptoms, and most are associated with intolerable side effects and/or serious potential adverse events such as liver toxicity or Stevens-Johnson Syndrome.

Therefore, there is a dire and currently unmet need for compositions and methods useful in treating patients diagnosed with a variety of distinct refractory epilepsy syndromes which are safe and effective. Further, there is a dire and currently unmet need for compositions and methods useful in preventing, treating or ameliorating seizures in a patient diagnosed with Doose syndrome.

### SUMMARY OF THE INVENTION

According to a first aspect of the present invention, there is provided a method of treating and/or preventing one or more symptoms of Doose syndrome in a patient comprising administering an effective dose of fenfluramine to the patient, as a monotherapy or in combination with one or more drugs as described herein, as well as a pharmaceutical composition for use in such a method, comprising a pharmaceutically acceptable carrier and fenfluramine or a pharmaceutically acceptable salt thereof.

According to another aspect of the present invention, there is provided a method of treating, preventing and/or ameliorating seizures in a patient diagnosed with Doose syndrome comprising administering an effective dose of fenfluramine or a pharmaceutically acceptable salt thereof to the patient, as well as a pharmaceutical composition for use in such a method, comprising a pharmaceutically acceptable carrier and fenfluramine or a pharmaceutically acceptable salt thereof.

According to yet another aspect of the present invention, there is provided a method of treating a patient that exhibits a mutation in one or more of a gene selected from the group consisting of CHD2 (15q26), GABRG2 (5q34), SCN1A (2q24.3), SCN1B (19q13.12), SLC2A1 (1p34.2), and SLC6A1 (3p25.3), by administering an effective dose of fenfluramine or a pharmaceutically acceptable salt thereof to the patient, as well as a pharmaceutical composition for use in such a method, comprising a pharmaceutically acceptable carrier and fenfluramine or a pharmaceutically acceptable salt thereof.

Still another aspect of this invention contemplates a method for stimulating one or more 5-HT receptors in the brain of a patient diagnosed with Doose syndrome by administering an effective dose of fenfluramine or a pharmaceutically acceptable salt thereof to the patient, as well as a pharmaceutical composition for use in such a method, comprising a pharmaceutically acceptable carrier and fenfluramine or a pharmaceutically acceptable salt thereof. Illustrative one or more 5-HT receptors are selected from the group consisting of one or more of 5-HT1A, 5-HT2A, 5-HT2B, 5-HT2C, 5-HT5A, and 5-HT7.

A further aspect of the invention provides a method for binding or modulating the activity of one or more of SERT (serotonin transporter), the NaV1.5 sodium channel subunit, the Sigma-1 receptor, the Sigma-2 receptor, the muscarinic M1 receptor, the β-adrenergic receptor, and the β2-adrenergic receptor in the brain of a patient diagnosed with Doose syndrome, by administering an effective dose of fenfluramine or a pharmaceutically acceptable salt thereof to the patient, as well as a pharmaceutical composition for use in such a method, comprising a pharmaceutically acceptable carrier and fenfluramine or a pharmaceutically acceptable salt thereof.

Yet a further aspect of the invention contemplates co-administration of an effective dose of one or more co-therapeutic agents with the fenfluramine or a pharmaceutically acceptable salt thereof in the methods and compositions for use provided herein, wherein the co-therapeutic agents can be selected from the group consisting of valproic acid, lamotrigine, levetiracetam, topiramate, zonisamide, rufinamide, clobazam, felbamate, ethosuximide, nitrazepam, adrenocorticotrophic hormone, methylprednisolone, prednisone, dexamethasone, clonazepam, clorazepate, perampanel, stiripentol, cannabidiol, and tetrahydrocannabinol. Use of a pharmaceutically acceptable salt of a co-therapeutic agent is also contemplated.

A still further aspect of the invention provides a method of treating or preventing the symptoms of Doose syndrome in a patient diagnosed with Doose syndrome comprising administering an effective dose of fenfluramine or pharmaceutically acceptable salt thereof to the patient, wherein the dose is administered in an amount in the range of from about 10.0 mg/kg/day to about 0.01 mg/kg/day, such as from about 0.8 mg/kg/day to about 0.01 mg/kg/day, or administered at 120 mg/day or less; or 90 mg/day or less, or 60 mg/day or less or 30 mg/day or less, or 20 mg or less, or 10 mg or less, and can be administered in the absence of the administration of any other pharmaceutically active compound, as well as a pharmaceutical composition for use in such a method, comprising a pharmaceutically acceptable carrier and fenfluramine or a pharmaceutically acceptable salt thereof. In an aspect the dose is administered at 20 mg/day or less.

In a still further aspect of the invention, the method is carried out wherein the effective dose is administered in a form selected from the group consisting of oral, injectable, transdermal, buccal, inhaled, nasal, rectal, vaginal, or parental, and wherein the formulation is oral, the formulation can be liquid which can be a solution or a suspension can be present within a container closed with a cap connected to a syringe graduated to determine the volume extracted from the container wherein the volume extracted relates to the amount of fenfluramine in a given liquid volume of formulation e.g. one millimeter of formulation contains 2.5 mg of fenfluramine. In another aspect of the invention, the method is administered in a solid oral formulation in the form of a tablet, capsule, lozenge, or sachet.

The methods described herein can be carried out as a co-treatment with a different pharmaceutically active compound. The methods described herein can be carried out in a process wherein the patient is first subjected to a series of tests to confirm diagnoses of Doose syndrome.

A still further aspect of the invention provides a kit for treating Doose syndrome (e.g., for treating, preventing and/or ameliorating a symptom of Doose syndrome) in a patient diagnosed with Doose syndrome wherein the kit comprises: a formulation comprising a pharmaceutically acceptable carrier and an active ingredient comprising fenfluramine or a pharmaceutically acceptable salt thereof; and instructions for treating a patient diagnosed with Doose syndrome by administering the formulation to the patient. In yet another aspect, the fenfluramine is an oral liquid formulation or a solid oral dosage form or a transdermal patch; and the kit further comprises instructions for treating a patient diagnosed with Doose syndrome by administering the formulation to the patient.

In another aspect of the invention, the kit consists of a liquid oral formulation in a container and a calibrated syringe with instructions, wherein the amount of fenfluramine in the liquid dose is measured by reference to calibrations on the syringe, and include calibrations wherein a volume of solution equates to a known amount of fenfluramine such as about 1.25 mg, about 1.5 mg, about 1.75 mg, about 2.0 mg, about 2.25 mg, or about 2.5 mg.

In another aspect of the invention, the kit includes instructions relating to dosing the patient based on patient weight and volume of solution based on the concentration of fenfluramine in the solution.

Another aspect of the invention is a use of a fenfluramine composition in treating, preventing and/or ameliorating symptoms of Doose syndrome in a patient diagnosed with Doose syndrome which use can include placing the fenfluramine in a liquid solution and withdrawing that liquid solution into a graduated syringe.

An aspect of the invention includes a method of treating, preventing and/or ameliorating symptoms of Doose syndrome in a patient diagnosed with Doose syndrome, comprising: administering an effective dose of fenfluramine or a pharmaceutically acceptable salt thereof to the patient, as well as a pharmaceutical composition for use in such a method, comprising a pharmaceutically acceptable carrier and fenfluramine or a pharmaceutically acceptable salt thereof.

In another aspect of the invention the patient exhibits one or more mutations in one or more of a gene selected CHD2 (15q26), GABRG2 (5q34), SCN1A (2q24.3), SCN1B (19q13.12), SLC2A1 (1p34.2), and SLC6A1(3p25.3).

In another aspect of the invention the fenfluramine is adjunctively administered with an additional pharmaceutically active drug.

In another aspect of the invention as described here the fenfluramine is the only pharmaceutically active drug administered to the patient for treating symptoms of Doose syndrome.

In another aspect of the invention as described here the symptoms are seizures.

In another aspect of the invention as described here the fenfluramine is administered in an amount of from 10.0 mg/kg/day to 0.01 mg/kg/day and wherein the fenfluramine is administered a dosage form selected from the group consisting of oral, injectable, transdermal, inhaled, nasal, rectal, vaginal or parenteral delivery; or wherein the fenfluramine is administered in an amount of from 0.8 mg/kg/day to 0.01 mg/kg/day.

In another aspect of the invention as described here the fenfluramine is in an oral solution in an amount selected from the group consisting of 120 mg or less, 60 mg or less, 30 mg or less, 20 mg or less and 10 mg or less. In aspect, the fenfluramine is in an oral solution in an amount of 20 mg or less.

In another aspect of the invention as described here the dosage form consists essentially only of fenfluramine as the active ingredient.

The invention further includes, on an aspect, administering a co-therapeutic agent selected from the group consisting of valproic acid, lamotrigine, levetiracetam, topiramate, zonisamide, rufinamide, clobazam, felbamate, ethosuximide, nitrazepam, adrenocorticotrophic hormone, methylprednisolone, prednisone, dexamethasone, clonazepam, clorazepate, perampanel, stiripentol, cannabidiol, and tetrahydrocannabinol, and pharmaceutically acceptable salts and bases thereof.

The invention further includes, in an aspect determining that a subject exhibits a mutation in a gene selected CHD2 (15q26), GABRG2 (5q34), SCN1A (2q24.3), SCN1B (19q13.12), SLC2A1 (1p34.2), and SLC6A1(3p25.3); and administering a therapeutically effective amount of fenfluramine or a pharmaceutically acceptable salt thereof to the subject and thereby preventing and/or ameliorating seizures in the subject.

The invention also includes a method of stimulating 5-HT receptors in a patient diagnosed with Doose syndrome, comprising: administering an effective dose of fenfluramine or a pharmaceutically acceptable salt thereof to the patient, as well as a pharmaceutical composition for use in such a method, comprising a pharmaceutically acceptable carrier and fenfluramine or a pharmaceutically acceptable salt thereof.

The invention also includes a kit for treating a symptom of Doose syndrome in a patient diagnosed with Doose syndrome, comprising: a container comprising a plurality of doses of a formulation comprising a pharmaceutically acceptable carrier and an active ingredient comprising fenfluramine or a pharmaceutically acceptable salt thereof; and instructions for treating the patient diagnosed with Doose syndrome by withdrawing the formulation from the container, and administering the formulation to the patient. Optionally, in the kit the formulation is either (a) an oral solution of a fenfluramine salt at a concentration of 2.5 mg/ml or (b) an oral solution of a fenfluramine base at a concentration of 2.2 mg/ml; and the instructions indicate dosing the patient based on patient weight and volume of oral solution administered.

The invention also includes a kit, wherein the formulation is a solid oral formulation selected from the group consisting of: a tablet, a disintegrating table, a capsule, a lozenge, and a sachet wherein the fenfluramine is present in the formulation in an amount of from 5 mg to 120 mg.

The invention also includes a kit, wherein said formulation is provided in a transdermal patch.

The invention also includes a kit, wherein the formulation is a liquid formulation for oral administration.

The invention also includes a kit, wherein the formulation consists essentially only of fenfluramine as the sole pharmaceutically active ingredient.

All aspects of the invention may include administering the effective dose after a meal of a ketogenic diet, before the ketogenic meal or while the patient is maintained on a ketogenic diet.

An aspect of the invention is a use of a pharmaceutical formulation in treating, preventing or ameliorating symptoms of Doose syndrome in a subject, the formulation comprising a therapeutically effective amount of fenfluramine or a pharmaceutically acceptable salt thereof, and a pharmaceutically acceptable carrier.

An aspect of the invention is a use of fenfluramine or a pharmaceutically acceptable salt thereof in the manufacture of a medicament for treating, preventing or ameliorating symptoms of Doose syndrome in a subject.

An aspect of the invention is a formulation of fenfluramine or a pharmaceutically acceptable salt thereof for use in treating, preventing or ameliorating symptoms of Doose syndrome in a subject.

In specific aspects, the present invention provides:

[1] A pharmaceutical composition for use in treating, preventing and/or ameliorating symptoms of Doose syndrome in a patient diagnosed with Doose syndrome, comprising: a pharmaceutically acceptable carrier; and fenfluramine or a pharmaceutically acceptable salt thereof in an amount sufficient to treat, prevent and/or ameliorate said symptoms of Doose in the patient.
[2] The composition for use of [1], wherein the patient exhibits one or more mutations in one or more of a gene selected from CHD2 (15q26), GABRG2 (5q34), SCN1A (2q24.3), SCN1B (19q13.12), SLC2A1 (1p34.2), and SLC6A1(3p25.3).
[3] The composition for use of [1] or [2] wherein fenfluramine is for use adjunctively with an additional pharmaceutically active drug.
[4] The composition for use of [1] or [2] wherein the fenfluramine is for use as the only pharmaceutically active drug in said treating, preventing and/or ameliorating symptoms of Doose syndrome.
[5] The composition for use of any one of [1] to [4], wherein the symptoms are seizures.
[6] The composition for use of any one of [1] to [5], wherein: (a) said treating, preventing and/or ameliorating comprises administering an amount of fenfluramine of from 10.0 mg/kg/day to 0.01 mg/kg/day or from 0.8 mg/kg/day to 0.01 mg/kg/day; or (b) said treating, preventing and/or ameliorating comprises administering the fenfluramine in a dosage form selected from the group consisting of oral, injectable, transdermal, inhaled, nasal, rectal, vaginal or parenteral delivery; or (c) said treating, preventing and/or ameliorating comprises administering an amount of fenfluramine of from 10.0 mg/kg/day to 0.01 mg/kg/day, or from 0.8 mg/kg/day to 0.01 mg/kg/day, in a dosage form selected from the group consisting of oral, injectable, transdermal, inhaled, nasal, rectal, vaginal or parenteral delivery.
[7] The composition for use of any one of [1]-[6], wherein: (a) the composition is in an oral solution comprised of fenfluramine in an amount selected from the group consisting of 120 mg or less, 60 mg or less, 30 mg or less, 20 mg or less and 10 mg or less; or (b) the composition is in an oral solution comprised of fenfluramine in an amount of 20 mg or less.
[8] The composition for use as defined in any one of [1]-[3] and [5]-[7], wherein said treating, preventing and/or ameliorating further comprises: administering a co-therapeutic agent selected from the group consisting of valproic acid, lamotrigine, levetiracetam, topiramate, zonisamide, rufinamide, clobazam, felbamate, ethosuximide, nitrazepam, adrenocorticotrophic hormone, methylprednisolone, prednisone, dexamethasone, clonazepam, clorazepate, perampanel, stiripentol, cannabidiol, and tetrahydrocannabinol, and pharmaceutically acceptable salts and bases thereof.
[9] A composition for use in stimulating 5-HT receptors in a patient diagnosed with Doose syndrome, wherein the patient exhibits a mutation in a gene selected CHD2 (15q26), GABRG2 (5q34), SCN1A (2q24.3), SCN1B (19q13.12), SLC2A1 (1p34.2), and SLC6A1(3p25.3), comprising an effective dose of fenfluramine or a pharmaceutically acceptable salt thereof.
[10] The composition for use of any one of [1]-[9], wherein the patient is on a ketogenic diet.
[11] A kit for treating, preventing and/or ameliorating a symptom of Doose syndrome in a patient diagnosed with Doose syndrome, comprising: a container comprising a plurality of doses of a formulation comprising a pharmaceutically acceptable carrier and an active ingredient comprising fenfluramine or a pharmaceutically acceptable salt thereof; and instructions for treating the patient diagnosed with Doose syndrome by withdrawing the formulation from the container, and administering the formulation to the patient.
[12] The kit as defined in [11], wherein: the formulation is either (a) an oral solution of a fenfluramine salt at a concentration of 2.5 mg/ml or (b) an oral solution of a fenfluramine base at a concentration of 2.2 mg/ml; and the instructions indicate dosing the patient based on patient weight and volume of oral solution administered.
[13] The kit as defined in [11], wherein the formulation is a solid oral formulation selected from the group consisting of: a tablet, a disintegrating table, a capsule, a lozenge, and a sachet; wherein the fenfluramine is present in the formulation in an amount of from 5 mg to 120 mg.
[14] The kit as defined in [11], wherein said formulation is provided in a transdermal patch.
[15] The kit of any one of [11] or [12], wherein the formulation is a liquid formulation for oral administration.

These and other objects, advantages, and features of the invention will become apparent to those persons skilled in the art upon reading the details of the methods of treating symptoms of Doose syndrome as more fully described below.

### BRIEF DESCRIPTION OF THE DRAWINGS

The present disclosure is best understood from the following detailed description when read in conjunction with the accompanying drawings. Included in the drawings are the following FIGURES.

FIG. 1 shows a flow chart depicting the patient visit and dosage titration algorithm using in the clinical trial described in Example 1 herein. Shown are the procedures followed during each of the patient visits and the scheme for fenfluramine dosage titration for non-responding patients.

### DETAILED DESCRIPTION OF THE INVENTION

### Definitions

Before the present method, compositions, formulations, and kits are described, it is to be understood that the invention provided by the present disclosure is not limited to the particular embodiments described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference, to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a seizure” includes a plurality of such seizures and reference to “the formulation” includes reference to one or more formulations and equivalents thereof known to those skilled in the art, and so forth.

To avoid doubt, the term “prevention” of seizures means the total or partial prevention (inhibition) of seizures. Ideally, the methods of the present invention result in a total prevention of seizures. However, the invention also encompasses methods in which the instances of seizures are decreased in frequency by at least 40%, at least 50%, at least 55%, at least 60%, at least 65%, at least 70%, at least 75%, at least 80%, at least 85%, or at least 90%. In addition, the invention also encompasses methods in which the instances of seizures are decreased in duration or severity by at least 40%, at least 50%, at least 55%, at least 60%, at least 65%, at least 70%, at least 75%, at least 80%, at least 85%, or at least 90%.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

After extensive research, it has unexpectedly been found that fenfluramine can be used to treat Doose syndrome or prevent or decrease the frequency and/or severity of its symptoms.

Without being bound by theory, fenfluramine has been known to trigger the release of serotonin (5-HT) in the brain due to disruption of its vesicular storage, and to inhibit serotonin reuptake, by increasing vesicular sequestration of SERT and concomitantly decreasing SERT transport of serotonin. See Rothman et al., “High-Dose Fenfluramine Administration Decreases Serotonin Transporter Binding, but Not Serotonin Transporter Protein Levels, in Rat Forebrain” Synapse 50:233-239 (2003). However, until the present invention was made, it was not known that fenfluramine's mechanism of action makes it suitable for the treatment of Doose syndrome, nor have any prior scientific publications demonstrated or proposed that 5-HT abnormalities are a possible underlying pathophysiologic cause for Doose syndrome, or otherwise causally related to the associated seizures in this specific epilepsy condition. Furthermore, reflective of the absence of any scientific hypothesis relating serotonin abnormalities in Doose syndrome, there are no studies nor even individual case reports in the medical literature which describe attempts to treat Doose syndrome using medications that interact with serotonin. The lack of any data or even speculation in the literature regarding the use of serotonergic agents in general or fenfluramine in particular to treat Doose syndrome underscore the unexpected nature of this invention: given that Doose syndrome is a devastating refractory epilepsy condition and the number of people affected, investigators would be strongly motivated to investigate any treatment they perceived as having any potential for efficacy.

Thus, according to one aspect of the invention, the disclosure provides a novel method of treating, preventing or ameliorating seizures in a patient diagnosed with Doose syndrome by administering an effective dose of fenfluramine or a pharmaceutically acceptable salt thereof to the patient.

According to a further aspect of the present invention, the disclosure herein provides a method of treating or preventing seizures in a patient diagnosed with Doose syndrome by stimulating one or more 5-HT receptors in the brain of said patient, said one or more 5-HT receptors being selected from one or more of 5-HT1A, 5-HT2A, 5-HT2B, 5-HT2C, 5-HT5A, and 5-HT7. In various embodiments, the method comprises administering an effective dose of fenfluramine or a pharmaceutically acceptable salt thereof to the patient.

More recently, further investigation by the inventors into fenfluramine's mechanism of action has yielded a more complete understanding. Without being bound by theory, fenfluramine has been found to be active at other receptors in addition to the 5-HT receptors referenced above, including but not limited to activity at the NaV1.5 sodium channel subunit, the Sigma-1 receptor, the Sigma-2 receptor, the muscarinic M1 receptor, the β-adrenergic receptor, and the β2-adrenergic receptor, either directly or via downstream modulation of its effects. See U.S. Provisional Pat. App. No. 62/402,881 incorporated in its entirety herein.

Prior to their work, no reports of fenfluramine's activity at any of the NaV1.5 sodium channel alpha subunit, the Sigma-1 receptor, the Sigma-2 receptor, the muscarinic M1 receptor, the β-adrenergic receptor, or the β2-adrenergic receptor existed in the scientific literature, nor were there reports demonstrating or even hypothesizing that agents which exhibit activity with respect to one or more of those receptors are useful for treating, preventing, or ameliorating symptoms associated with Doose syndrome, nor for treating, preventing, or ameliorating epilepsy seizures in patients diagnosed with Doose syndrome.

Therefore, according to a still further aspect of the invention, there is provided herein a method of treating, preventing, or ameliorating seizures in a patient diagnosed with Doose syndrome by modulating the activity of one or more of SERT (serotonin transporter), the NaV1.5 sodium channel alpha subunit, the Sigma-1 receptor, the Sigma-2 receptor, the muscarinic M1 receptor, the β-adrenergic receptor, or the β2-adrenergic receptor in the brain of said patient. In various embodiments, the method comprises administering an effective dose of one or more of an agent active at one or more of the NaV1.5 sodium channel alpha subunit, the Sigma-1 receptor, the Sigma-2 receptor, the muscarinic M1 receptor, the β-adrenergic receptor, or the β2-adrenergic receptor. In various embodiments, agent is fenfluramine or a pharmaceutically acceptable salt thereof.

As mentioned above, genetics appears to play an important role in the etiology of Doose syndrome, including CHD2 (15q26), GABRG2 (5q34), SCN1A (2q24.3), SCN1B (19q13.12), SLC2A1 (1p34.2), and SLC6A1(3p25.3).

Thus, according to another aspect of the invention, the disclosure provides a method of treating a patient that exhibits a mutation in one or more of a gene selected from the group consisting of CHD2 (15q26), GABRG2 (5q34), SCN1A (2q24.3), SCN1B (19q13.12), SLC2A1 (1p34.2), and SLC6A1(3p25.3), by administering an effective dose of fenfluramine or a pharmaceutically acceptable salt thereof to the patient. In various embodiments, the method comprises administering fenfluramine or a pharmaceutical salt thereof to said patients exhibiting mutations the aforementioned genes to treat, prevent, or ameliorate seizures.

The methods, formulations and kits provided by the present disclosure can be employed in treating any appropriately diagnosed patient. In one aspect, the patient is an adult. In one aspect, the patient is 18 years old or less. In alternate exemplary embodiments of this aspect, the patient is aged about 18 or less, about 17 or less, about 16 or less, about 15 or less, about 14 or less, about 13 or less, about 12 or less, about 11 or less, about 10 or less, about 9 or less, about 8 or less, about 7 or less, about 6 or less, about 5 or less, or about 4 or less to about 0 months or more, about 1 month or more, about 2 months or more, about 4 months or more, about 6 months or more, or about 1 year or more. In exemplary embodiments of the methods, formulations and kits provided herein, the diagnosed patient is about one month old to about 18 years old when treated.

### Dosage

In embodiments of the methods, formulations, and kits provided by the present disclosure, any effective dose of fenfluramine can be employed. However, surprisingly low doses of fenfluramine have been found by the inventors to be effective, particularly for inhibiting or eliminating seizures in Doose syndrome patients. In general, the smallest dose which is effective for the particular patient should be used. While dosing is determined based on the needs of individual patients, doses effective in treating, preventing or ameliorating symptoms associated with Doose patients in patients diagnosed with the disease are generally well below the dosing used in weight loss.

Thus in some cases, in preferred embodiments of the methods provided by the present disclosure, a daily dose of less than about 10 mg/kg/day, such as less than about 9.5 mg/kg/day, less than about 9 mg/kg/day, less than about 8.5 mg/kg/day, less than about 8 mg/kg/day, less than about 7.5 mg/kg/day, less than about 7 mg/kg/day, less than about 6.5 mg/kg/day, less than about 6 mg/kg/day, less than about 5.5 mg/kg/day, less than about 5 mg/kg/day, less than about 4 mg/kg/day, less than about 4.5 mg, less than about 3.0 mg/kg/day, less than about 3.0 mg/kg/day, less than about 2.0 mg/kg/day, less than about 2.5 mg/kg/day, less than about 2.0 mg/kg/day, less than about 1.5 mg/kg/day, less than about 1.0 mg/kg/day,

such as less than about 1.0 mg/kg/day, less than about 0.95 mg/kg/day, less than about 0.9 meg/kg/day, less than about 0.85 mg/kg/day, less than about 0.85 mg/kg/day, less than about 0.8 mg/kg/day, less than about 0.75 mg/kg/day, less than about 0.7 mg/kg/day, less than about 0.65 mg/kg/day, less than about 0.6 mg/kg/day, less than about 0.55 mg/kg/day, less than about 0.5 mg/kg/day, less than about 0.45 mg/kg/day, less than about 0.4 mg/kg/day, less than about 0.35 mg/kg/day, less than about 0.3 mg/kg/day, less than about 0.25 mg/kg/day, less than about 0.2 mg/kg/day, less than about 0.15 mg/kg/day less than about 0.1 mg/kg/day,

such as less than about 0.075 mg/kg/day, less than about 0.05 mg/kg/day, less than about 0.025 mg/kg/day,

such as less than about 0.0225 mg/kg/day, less than about 0.02 mg/kg/day, less than about 0.0175 mg/kg/day, less than about 0.015 mg/kg/day, less than about 0.0125 mg/kg/day, or less than about 0.01 mg/kg/day, is employed.

Put differently, a preferred dose is less than about 10 to about 0.01 mg/kg/day. In some cases the dose is less than about 10.0 mg/kg/day to about 0.01 mg/kg/day, such as less than about 9.5 mg/kg/day to about 0.01 mg/kg/day, less than about 9.0 mg/kg/day to about 0.01 mg/kg/day, less than about 8.5 mg/kg/day to about 0.01 mg/kg/day, less than about 8.0 mg/kg/day to about 0.01 mg/kg/day, less than about 7.5 mg/kg/day to about 0.01 mg/kg/day, less than about 7.0 mg/kg/day to about 0.01 mg/kg/day, less than about 6.5 mg/kg/day to about 0.01 mg/kg/day, less than about 6.0 mg/kg/day to about 0.01 mg/kg/day, less than about 5.5 mg/kg/day to about 0.01 mg/kg/day, less than about 5.0 mg/kg/day to about 0.01 mg/kg/day, less than about 4.5 mg/kg/day to about 0.01 mg/kg/day, less than about 4.0 mg/kg/day to about 0.01 mg/kg/day, less than about 3.5 mg/kg/day to about 0.01 mg/kg/day, less than about 3.0 mg/kg/day to about 0.01 mg/kg/day, less than about 2.5 mg/kg/day to about 0.01 mg/kg/day, less than about 2.0 mg/kg/day to about 0.01 mg/kg/day, less than about 1.5 mg/kg/day to about 0.01 mg/kg/day, less than about 1.0 mg/kg/day to 0.01 mg/kg/day,

such as less than about 0.9 mg/kg/day to about 0.01 mg/kg/day, less than about 0.8 mg/kg/day to about 0.01 mg/kg/day, less than about 0.7 mg/kg/day to about 0.01 mg/kg/day, less than about 0.6 mg/kg/day to about 0.01 mg/kg/day, less than about 0.5 mg/kg/day to about 0.01 mg/kg/day, less than about 0.4 mg/kg/day to about 0.01 mg/kg/day, less than about 0.3 mg/kg/day to about 0.01 mg/kg/day, less than about 0.2 mg/kg/day to about 0.01 mg/kg/day or less than about 0.1 mg/kg/day to about 0.01 mg/kg/day.

As indicated above the dosing is based on the weight of the patient. However, for convenience the dosing amounts can be preset such as in the amount of 1.0 mg, 2.5 mg, 5 mg, 10 mg, 15 mg, 20 mg, 30 mg, or 60 mg. In certain instances, the dosing amount can be preset such as in the amount of about 0.25 mg to about 5 mg, such as about 0.25 mg, about 0.5 mg, about 0.75 mg, about 1.0 mg, about 1.25 mg, about 1.5 mg, about 1.75 mg, about 2.0 mg, about 2.25 mg, about 2.5 mg, about 2.75 mg, about 3.0 mg, about 3.25 mg, about 3.5 mg, about 3.75 mg, about 4.0 mg, about 4.25 mg, about 4.5 mg, about 4.75 mg, or about 5.0 mg.

The dosing amounts described herein can be administered one or more times daily to provide for a daily dosing amount, such as once daily, twice daily, three times daily, or four or more times daily, etc.,

In certain embodiments, the dosing amount is a daily dose of about 120 mg or less, such as about 120 mg, about 110 mg, about 100 mg, about 90 mg, about 80 mg, about 70 mg, about 60 mg, about 50 mg, about 40 mg, about 30 mg,

such as about 29 mg, about 28 mg, about 27 mg, about 26 mg, about 25 mg, about 24 mg, about 23 mg, about 22 mg, about 21 mg, about 20 mg, about 19 mg, about 18 mg, about 17 mg, about 16 mg, about 15 mg, about 14 mg, about 13 mg, about 12 mg, about 11 mg, about 10 mg, about 9 mg, about 8 mg, about 7 mg, about 6 mg, about 5 mg, about 4 mg, about 3 mg, about 2 mg, or about 1 mg. In some cases, the dose is generally well below the dosing used in weight loss.

The dose of fenfluramine to be used in methods provided by the present disclosure can be provided in the form of a kit, including instructions for using the dose in one or more of the methods provided herein. Such kits are described infra.

### Formulations and Administration

Fenfluramine for use in the methods, formulations, and kits of the present disclosure can be produced according to any pharmaceutically acceptable process known to those skilled in the art. Examples of processes for synthesizing fenfluramine are provided in the following patent documents: GB1413070, GB1413078 and EP441160.

Fenfluramine can be administered in the form of the free base, or in the form of a pharmaceutically acceptable salt, for example selected from the group consisting of hydrochloride, hydrobromide, hydroiodide, maleate, sulphate, tartrate, acetate, citrate, tosylate, succinate, mesylate and besylate. Further illustrative pharmaceutically acceptable salts can be found in Berge et al., J. Pharm Sci. (1977) 68(1): 1-19.

The dose of fenfluramine administered according to the methods of the present disclosure can be administered systemically or locally. Methods of administration can include administration via enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, it can be desirable to administer one or more compounds of the disclosure locally to the area in need of treatment. This can be achieved, for example, by local infusion during, topical application, by injection, by means of a catheter, by means of a suppository, or by means of an implant, said implant being of a porous, non-porous, or gelatinous material, including membranes, such as sialastic membranes, or fibers.

The dose of fenfluramine administered in the methods of the present disclosure can be formulated in any pharmaceutically acceptable dosage form including, but not limited to (a) oral dosage forms such as tablets including orally disintegrating tablets, capsules, and lozenges, oral solutions or syrups, oral emulsions, oral gels, oral films, buccal liquids, powder e.g. for suspension, and the like; (b) injectable dosage forms; (c) transdermal dosage forms such as transdermal patches, ointments, creams; (c) inhaled dosage forms; and/or (e) nasally, (f) rectally, and (g) vaginally administered dosage forms.

Such dosage forms can be formulated for once a day administration, or for multiple daily administrations (e.g. 2, 3 or 4 times a day administration). Alternatively, for convenience, dosage forms can be formulated for less frequent administration (e.g., monthly, bi-weekly, weekly, every fourth day, every third day, or every second day), and formulations which facilitate extended release are known in the art.

The dosage form of fenfluramine employed in the methods, formulations, and kits provided by the present disclosure can be prepared by combining fenfluramine or a pharmaceutically acceptable salt thereof in a formulation with one or more pharmaceutically acceptable diluents, carriers, adjuvants, and the like in a manner known to those skilled in the art of pharmaceutical formulation.

In some embodiments, formulations suitable for oral administration can include (a) liquid solutions or syrups, such as an effective amount of the compound dissolved in diluents, such as water, or saline; (b) capsules, sachets or tablets, each containing a predetermined amount of the active ingredient (fenfluramine), as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

For an oral solid pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition can be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition can also contain minor amounts of non-toxic auxiliary substances such as wetting agents, emulsifying agents, or buffers.

By way of illustration, the fenfluramine composition can be admixed with conventional pharmaceutically acceptable carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1% to about 90% by weight of the active compound, and more generally from about 1% to about 30% by weight of the active compound. The pharmaceutical compositions can contain common carriers and excipients, such as corn starch or gelatin, lactose, dextrose, sucrose, microcrystalline cellulose, kaolin, mannitol, dicalcium phosphate, sodium chloride, and alginic acid. Disintegrators commonly used in the formulations of this disclosure include croscarmellose, microcrystalline cellulose, corn starch, sodium starch glycolate and alginic acid.

Formulations suitable for topical administration can be presented as creams, gels, pastes, or foams, containing, in addition to the active ingredient, such carriers as are appropriate. In various embodiments, the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo- (ethylene oxide-) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations can further include other components, e.g., astringents, fragrances, pigments, skin penetration enhancing agents, sunscreens (e.g., sun blocking agents), etc.,

Particular formulations of the formulations provided herein are in an oral liquid form. The liquid can be a solution or suspension and can be an oral solution or syrup, which is included in a bottle with a syringe graduated in terms of milligram or milliliter amounts which will be obtained in a given volume of solution. The liquid solution makes it possible to adjust the volume of solution for appropriate dosing of small children, who can be administered fenfluramine in an amount anywhere from 1.25 mg to 30 mg and any amount between, in 0.25 milligram or larger increments, and thus administered in amounts of 1.25 mg, 1.5 mg, 1.75 mg, 2.0 mg, etc., such as about 1.25 mg or more, about 1.5 mg or more, about 1.75 mg or more, about 2.0 mg or more, about 2.25 mg or more, about 2.5 mg or more, about 2.75 mg or more, about 3.0 mg or more, about 3.25 mg or more, about 3.75 mg or more, about 4.0 mg or more, about 4.25 mg or more, about 4.5 mg or more, about 5.0 mg or more, about 5.25 mg or more, about 5.5 mg or more, about 5.75 mg or more, about 6.0 mg or more, about 6.25 mg or more, about 6.5 mg or more, about 6.75 mg or more, about 7 mg or more, about 7.25 mg or more, about 7.5 mg or more, about 7.75 mg or more, about 8.0 mg or more, about 8.25 mg or more, about 8.5 mg or more, about 8.75 mg or more, about 9.0 mg or more, about 9.25 mg or more, about 9.5 mg or more, about 9.75 mg or more, about 10 mg or more, about 10.25 mg or more, about 10.5 mg or more, about 10.75 mg or more, about 11.0 mg or more, about 11.25 mg or more, about 11.5 mg or more, about 11.75 mg or more, about 12.0 mg or more, about 12.25 mg or more, about 12.5 mg or more, about 12.75 mg or more, about 13.0 mg or more, about 13.25 mg or more, about 13.5 mg or more, about 13.75 mg or more, about 14.0 mg or more, about 14.25 mg or more, about 14.5 mg or more, about 14.75 mg about 15.0 mg or more,

such as about 15.5 mg or more, about 16.0 mg or more, about 16.5 mg or more, about 17 mg or more, about 17.5 mg or more, about 18.0 mg or more, about 18.5 mg or more, about 19 mg or more, about 19.5 mg or more,

such as about 20 mg or more, about 21 mg or more, about 22 mg or more, about 23 mg or more, about 24 mg or more, about 25 mg or more, about 26 mg or more, about 27 mg or more, about 28 mg or more, about 29 mg or more, or about 30 mg.

### Co-Therapies

A specific aspect of the methods, formulations and kits provided herein is a treatment carried out to relieve symptoms of Doose syndrome by the administration of only fenfluramine. However, the fenfluramine can also be co-administered with other known pharmaceutical drugs such as a co-therapeutic agent selected from the group consisting of valproic acid, lamotrigine, levetiracetam, topiramate, zonisamide, rufinamide, clobazam, felbamate, ethosuximide, nitrazepam, adrenocorticotrophic hormone, methylprednisolone, prednisone, dexamethasone, clonazepam, clorazepate, perampanel, stiripentol, cannabidiol, and tetrahydrocannabinol, and a pharmaceutically acceptable salt or base thereof.

The pharmaceutical drugs referred to above have recommended dosing amounts. Those recommended dosing amounts are provided within the most current version of the Physician's Desk Reference (PDR) or http://emedicine.medscape.com/ both of which are incorporated herein by reference specifically with respect to the co-therapeutic agents listed above and more specifically with respect to the dosing amounts recommended for those drugs.

Co-therapeutic agents useful in the methods, formulations and kits described herein can be used in the recommended dosing amount or can be used in a range of from 1/100th to 100 times, 1/10th to 10 times, ⅕ to 5 times, or ½ to twice the recommended dosing amount, or any incremental 1/100th amount in between those ranges.

### Kits

In one aspect, the disclosure provides a kit for treating and or preventing symptoms of Doose syndrome in a patient diagnosed with Doose syndrome, the kit comprising:

a container holding a liquid formulation of fenfluramine; and

instructions for administering the liquid formulation to a patient in order to treat Doose syndrome.

In a further aspect, the kit additionally comprises:

a device for withdrawing the liquid formulation from the container and dispensing it for administration to a patient.

In various embodiments, the device can be a calibrated syringe or graduated pipette useful for delivering varying doses of the fenfluramine liquid. In one embodiment, the dispensing device is a metered dose dispenser capable of dispensing a predetermined volume of fenfluramine liquid. In one embodiment, the metered dose dispenser can be adjusted to dispense different volumes of fenfluramine liquid, providing for convenient, consistent, and accurate dosing.

The formulation can be a solution or suspension and is prepared such that a given volume of the formulation contains a known amount of active fenfluramine.

In one embodiment, the dispenser is a syringe connected to the container and configured to withdraw the liquid formulation from the container, wherein the syringe is marked with levels of graduation noting volume of formulation withdrawn. or a metered dose dispenser calibrated to deliver a predetermined volume of the fluid, which dispenser can be adjusted to deliver different volumes of liquid.

In certain embodiments, the kit can comprise a dosage form comprising one or more co-therapeutic agents.

In methods which employ the kits provided by the present disclosure, fenfluramine can be employed as a monotherapy in the treatment of Doose syndrome. Alternatively, fenfluramine can be co-administered in combination with one or more pharmaceutically active agents, which can be provided together with the fenfluramine in a single dosage formulation, or separately, in one or more separate pharmaceutical dosage formulations. Where separate dosage formulations are used, the subject composition and one or more additional agents can be provided as part of the kit, or separately, and can be administered concurrently, or at separately staggered times, i.e., sequentially. Co-therapeutic agents suitable for use in kits are described above. Use of a pharmaceutically acceptable salt of a co-therapeutic agent is also contemplated.

### EXAMPLES

The invention is further illustrated in the following Examples.

### Example 1

### Safety and Efficacy of Fenfluramine Hydrochloride Oral Solution in Pediatric Doose Syndrome Patients

The efficacy of fenfluramine as an add-on treatment in children diagnosed with mycolonic atonic epilepsy (Doose syndrome) is studied in a Phase 2 Clinical Trial.

### Trial Objectives, Design, and Overview

An open-label, non-randomized non-placebo controlled add-on study is designed to assess the efficacy and safety of low-dose add-on fenfluramine on children diagnosed with myoclonic atonic epilepsy (Doose syndrome) experiencing seizures refractory to standard therapies. Oral formulations of fenfluramine are administered across a range of fenfluramine doses (0.2, 0.4, and 0.8 mg/kg/day, to a maximum of 30 mg/day). The trial is conducted over a 14-week period with responders eligible for participation in an open-label extension. Parents/caregivers use a daily diary to record the number/type of seizures, dosing, and use of rescue medication.

The 6-week Baseline Period consists of the establishment of initial eligibility during a screening visit followed by a 6-week observation period during which time subjects will be assessed for baseline seizure activity based on recordings of daily seizure activity entered into a diary. Upon completion of the Baseline Period, subjects who qualify for the study are initiated on fenfluramine at 0.4 mg/kg/day (maximum dose 30 mg/day).

After 4 weeks, the patient is examined for efficacy and tolerability. In patients who tolerate the medication but are not free of convulsive seizures, and are receiving a total daily dose of less than 30 mg, the dose is increased to 0.8 mg/kg/day (maximum dose 30 mg/day). Patients who are seizure free, continue to receive 0.4 mg/kg/day. Patients who do not tolerate fenfluramine receive a reduced dose of 0.2 mg/kg/day.

After an additional 4 weeks, the patient is again examined for efficacy and tolerability. Patients completing the study can opt to continue to receive fenfluramine as an open label extension.

Children enrolled in the open label extension study will be assessed for tolerability and efficacy every 3 months. Those wishing to withdraw from the study will have fenfluramine tapered.

Patients are sourced by contacting providers who care for children with Myoclonic Atonic epilepsy (Doose) at the study site and surrounding child neurology clinics will be notified for recruitment.

### Study Endpoints

The endpoints of interest are: (1) determination and documentation of fenfluramine's efficacy as adjunctive therapy by documenting percent reduction in convulsive seizures and drop seizures in subjects taking fenfluramine compared to baseline; (2) determination and documentation of fenfluramine's minimum effective dose for both seizure freedom and ≥50% reduction in convulsive or drop seizures are documented at 0.2, 0.4, or 0.8 mg/kg/day; (3) determination of the longest convulsive or drop seizure-free interval while treated with fenfluramine compared to baseline during treatment with fenfluramine compared to baseline

### Inclusion and Exclusion Criteria

Patients are recruited via referrals from providers treating children at childhood epilepsy clinics at and near study sites. and selected for inclusion in the study according to criteria comprising a combination of age, physical and psychological characteristics, and resistance to treatment with conventional therapies. Details of selection criteria for each of the baseline and treatment portions of the study are provided in Table 3, Table 4, and Table 5 below.

TABLE 3
Inclusion Criteria (baseline portion of study)

| | |
|---|---|
| Study Subjects | Children age 2-8 years with a documented medical history supporting a |
| | clinical diagnosis of myoclonic atonic epilepsy (Doose syndrome) who |
| | are not seizure free with the treatment of >2 accepted therapies. |
| Accepted anti-epileptic medications | benzodiazepine (clonazepam, clorazepate, clobazam); |
| | Ethosuximide, Felbamate; Lamotrigine; Levetiracetam; Rufinamide; |
| | Topiramate; Valproic acid; Zonisamide; Perampanel |
| Accepted non-medication therapy | Ketogenic or modified Atkins diet |
| Seizure onset | age 2 to 8 years inclusive in otherwise healthy patient |
| Initial development | Normal |
| MRI | History of normal brain MRI without cortical brain malformation |
| EEG | History of EEG demonstrating generalized 2-3 Hz slow spike and wave |
| Lack of alternative diagnosis | |
| Seizures | >6 convulsive or drop seizures per 6-week period for 12 weeks prior to |
| | screening |
| | Convulsive seizures (Tonic-clonic, tonic, atonic, clonic) |
| | Drop seizures (Tonic, atonic, myoclonic atonic) |
| | Seizure frequency as reported by parent/guardian to investigator or |
| Stabilized primary | >4 weeks prior to screening |
| treatments/interventions | |
| Informed consent | subjects have been informed of nature of study |
| | informed consent obtained from legally responsible parent/guardian |
| | Subject's assent given per standards of Institutional Review (IRB) |
| | requirements, where subject is capable |
| Compliance with study requirements | Parent/guardian willing and capable to comply with requirements re: |
| | diary completion, visit schedule and study drug accountability |

TABLE 4
Exclusion Criteria (baseline portion of study)

| Disqualifying medical history | Findings suggesting alternative causes of seizures, including: | |
|---|---|---|
| | History of epileptic or infantile spasms | |
| | History of developmental delay prior to onset of seizures | |
| | MRI abnormalities causative of seizures | |
| | Diagnosis of GLUT-1 deficiency | |
| | Clinical history & findings consistent with Lennox-Gastaut | |
| | syndrome | |
| | Known hypersensitivity to fenfluramine or excipients used in the | |
| | study medication | |
| | Past or current history of glaucoma | |
| | Hepatic | Past or current history of severe hepatic impairment |
| | impairment | Asymptomatic patients with mild hepatic |
| | | impairment (elevated liver enzymes <3x ULN |
| | | and/or elevated bilirubin <2 ULN) may be included |
| | | at discretion of investigator and subject to review |
| | | and approval by IDSC in conjunction with the study |
| | | sponsor |

| | |
|---|---|
| | Clinically significant condition, relevant symptoms or significant |
| | illness <4 weeks prior to screening visit which negatively impacts |
| | study participation or data collection, or poses a risk to subject |
| Pre-existing conditions associated | Pulmonary arterial hypertension |
| with fenfluramine toxicity | Past or current history of cardiovascular disease |
| | (cardio valvulopathy, myocardial infarctions, or stroke) |
| | Past or current history of anorexia nervosa, bulimia, depression that |
| | required treatment (medical or psychological) for longer than 1 |
| | month |
| Disqualifying psychological | Subject at imminent risk of self-harm or harm to others, per opinion |
| conditions | of investigator based upon clinical interview and responses to C- |
| | SSRS). |
| | Automatic exclusion where Suicidal behavior w/in past 6 months |
| | per C-SSRS at screening or baseline including suicidal ideation |
| | with intent or plan |
| | Discretionary inclusion where suicidal ideation reported without |
| | specific plan, inclusion may be allowed at discretion of investigator |
| | conditioned upon documentation of rationale for inclusion and |
| | education of parent/guardian |
| Disqualifying concomitant | Centrally-acting anorectic agents |
| medications | MOIs |
| | Any centrally-acting compounds with clinically appreciable amount |
| | of serotonin agonist or antagonist properties (SSRIs; noradrenergic |
| | agonists e.g., atomoxetine; CYP450 2D6, 3A4 and/or 2B6 |
| | inhibitors or substrates) |
| | Carbamazepine, oxcarbazepine, eslicarbazepine, phenobarbital, or |
| | phenytoin |
| | (currently treated or within past 30 days) |
| | Positive results for urine THC panel OR Positive whole blood CBD |
| | at screening visit |
| Disqualifying Activities | Participation in another clinical trial within the past 30 days; |
| | Currently receiving another investigational drug |
| Refusal to comply with study | Dietary restrictions (grapefruit or Seville oranges for period starting |
| requirements | at beginning of baseline period and continuing throughout study) |
| | Study procedures (scheduled visits, drug administration plan, |
| | laboratory tests, other study procedures, and study restrictions |
| Refusal/inability to comply with | Scheduled visits, drug administration plan, laboratory tests, other |
| study requirements | study procedures, and study restrictions |

TABLE 5
Inclusion Criteria (treatment portion of study)

| | |
|---|---|
| Cardiovascular | No cardiovascular abnormality on echocardiogram |
| | (including trace mitral or aortic regurgitation) or |
| Pulmonary | No signs of pulmonary hypertension |
| Seizure activity | Stable baseline seizure frequency |
| | >6 convulsive or drop seizure during 6-week baseline |
| | Minimum two seizures in first three weeks |
| | Two seizures in second three weeks |
| Compliance with | >90% compliance |
| seizure diary | |

Once enrolled, subjects are disqualified and removed from the study after tapering in cases of serious adverse events or intolerance that persists at a dosage of 0.2 mg/kg/day, lack of efficacy at a dosage of 0.8 mg/kg/day (30 mg day maximum), or non-compliance with study requirements. Treatment is also stopped in the event of increased severity and frequency of seizures after discussion with the principle investigator. Patients may also withdraw voluntarily. Upon withdrawing, a safety examination (i.e., blood sampling and cardiac ultrasound) is performed and fenfluramine use is tapered for one week at 50% of end dosage and then withdrawn completely.

### Ethics and Regulatory Approvals

Trial conduct complies with the most recent version of the principles of the Declaration of Helsinki and the principles of GCP, and in accordance with all applicable regulatory requirements. The study protocol and related documents is subject to ethical review by all requisite authorities. Participants give written informed consent prior to their enrollment and participation in compliance with all applicable laws, regulations and ethical guidelines as required, and ICFs are retained at participating trial sites in accordance with all applicable regulatory agencies and laws. All information and data related to the Study and disclosed to the Participating Site and/or Study Investigator are treated as confidential and are not disclosed to third parties or used for any purpose other than the performance of the study. Data collection, processing and disclosure of personal data are all subject to compliance with applicable personal data protections and personal data processing requirements.

### Fenfluramine and Dose Titration

Oral fenfluramine solution (2.5 mg/ml or 5 mg/ml) is provided by Zogenix International Limited, a wholly owned subsidiary of Zogenix, Inc. to study patients. Starting dosage is 0.2 mg/kg/day BID; second step at 0.4 mg/kg/day BID; maximum dosage at 0.8 mg/kg/day BID or 30 mg/day BID, whichever is less. Labeled bottles containing the oral fenfluramine suspension are given to patients and controlled at each visit. Bottle labels are kept in individual patient files. Calculation of bottle number and control of labels are done at the trial's conclusion. Patient compliance is assessed by control of oral solution quantity at each visit and collection of seizure diary with notification of drug intake.

### Visit Schedule

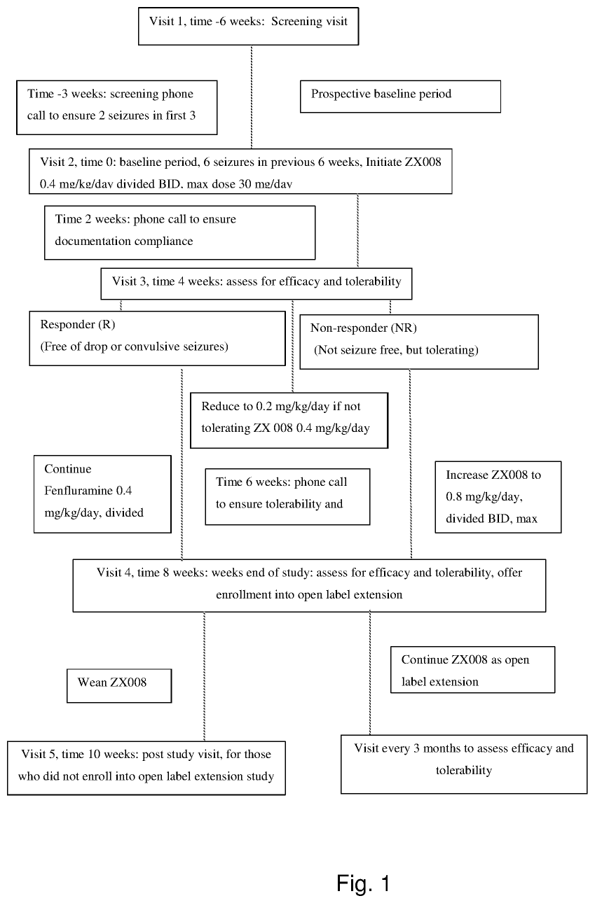

A flow chart illustrating the visit algorithm and fenfluramine titration scheme is shown in FIG. 1. Procedures followed during patient visits will now be described.

An initial screening visit occurs at day −42 to −40 to assess patients according to baseline period inclusion and exclusion criteria described above. Subsequently a screening phone call is made at day −21 to day −19 (“screening phone call”) to determine seizure activity during the period following the initial assessment.

Next, subject patients are assessed at a baseline visit on day 0 according to the Treatment Period Inclusion Criteria described above. Patients who satisfy those criteria and wish to participate in the trial are provided with information regarding trial particulars (e.g., adverse side effects, risks, etc.,), and written consent is obtained from a parent or guardian. Trial participants are then dosed with fenfluramine at 0.4 mg/kg/day (max dose 30 mg/day). Subsequently, patients receive a phone call at day 13-15 days (2-week phone call) to confirm documentation compliance.

At day 27-29, patients return for assessment of efficacy and tolerability. Where necessary, dose adjustments are made. Where 0.04 mg/kg/day is tolerated, but resolution of convulsive or drop seizures is not achieved, the dose is increased to 0.8 mg/kg/day (max dose 30 mg/day). If the dose of 0.4 mg/kg/day is not tolerated, then it is reduced to 0.2 mg/kg/day. Prior to that assessment, if there is significant seizure burden or side effects at the 2-week phone call, medication changes can be made at that time according to clinical discretion.

Subsequently, the patient is called a third phone call at day 41-43 (six-week phone call) to ensure continued tolerability and to document continued compliance.

Patients are then re-assessed at eight weeks. If there are significant side effects, and clinical discretion warrants urgent change, the dose can be decreased as follows: 0.8 to 0.4 mg/kg/day, or 0.4 to 0.2 mg/kg/day. If there are continued significant side effects to fenfluramine at 0.2 mg/kg/day and clinical discretion warrants urgent change, the patient will be withdrawn from the study.

The study is concluded at day 55-57. Patients who wish to continue receiving medication are enrolled in an open label extension visit, and are dosed at 0.2 to 0.8 mg/kg/day at their physician's discretion. Patients who do not enroll into the extension study are assessed at a final clinical visit on day 69-71.

Upon withdrawal, medication is tapered according to the schedule shown in Table 6 below.

TABLE 6
Dose Tapering Schedule

| | | Taper Step | | |
|---|---|---|---|---|
| | | Taper step 1 | Taper step 2 | Taper step 3 |
| Timepoint | | Day 1-4 | Day 5-8 | Day 9-12 |
| | | after study | after study | after study |
| | | completion | completion | completion |
| | | or early | or early | or early |
| | | termination | termination | termination |
| Dose | 0.2 mg/kg/day | discontinue | discontinue | n/a |
| | 0.4 mg/kg/day | 0.2 mg/kg/day | discontinue | n/a |
| | 0.8 mg/kg/day | 0.4 mg/kg/day | 0.2 mg/kg/day | discontinue |

Note:
maximum daily dose of fenfluramine is 30 mg

### Outcome Assessment Tools

Efficacy

Efficacy is assessed using the parameters shown in Table 7.

TABLE 7
Efficacy Assessment Parameters

| Parameter | Metric |
|---|---|
| Seizures | Number of seizures by type |
| | Convulsive seizure free interval |
| | Drop seizure free interval |
| | Impact of Pediatric Epilepsy Scale (Camfield et al, Epilepsia, 42(1): 104-112, 2001) |
| | Duration of prolonged seizures (seizure type that, during baseline, had duration >2 |
| | minutes) |
| | Number of episodes of status epilepticus |
| Seizure-related | Number of instances of rescue medication use and number of doses |
| interventions | Number of inpatient hospital admissions due to seizures |
| Clinical Impressions | Clinical Global Impression- Impression as assessed by principal investigator |
| | Global impression of sleep scale |
| | Improvement as assessed by parent/caregiver |

Safety

Adverse Events (AEs) are monitored, and reviewed by an independent Data Safety Committee. Assessment parameters include those listed in Table 8 below:

TABLE 8
Safety Parameters

| Parameter | Metric |
|---|---|
| Laboratory safety parameters | hematology, chemistry, urinalysis |
| Vital signs | blood pressure, heart rate, temperature, respiratory rate, body weight |

Physical examination
Neurological examination
12-lead ECGs
Doppler Echocardiograms

| | |
|---|---|
| Cognitive function | Tracked by administering The Vineland Adaptive Behavior Scales (Vineland- |
| | II, Sparrow SS, Cicchetti VD, Balia AD. Vineland adaptive behavior scales. |
| | 2nd edition American Guidance Service; Circle Pines, MN: 2005). |

Adverse Event Monitoring

Adverse events are monitored by the independent Data Safety Committee, comprised of three Board Certified Child and Adolescent Neurologists working at institutions other than Mayo Clinic

A separate International Pediatric Cardiology Advisory Board (IPCAB) will monitor the cardiac safety of the fenfluramine clinical trials

ECGs and Doppler echocardiograms will be centrally read (Biomedical Systems, Inc.) and interpreted under blinded conditions using pre-specified criteria, and if necessary, with review by the (IPCAB).

### Ketogenic Diet

In embodiments of the invention, any effective dose of fenfluramine can be employed with a ketogenic diet. In some cases, surprisingly low doses of fenfluramine have been found by the inventors to be efficacious, particularly for inhibiting or eliminating seizures in Doose syndrome patients. Thus, in preferred embodiments of the invention, the patient is on a ketogenic diet, and the maximum daily dose is not more than about 26 mg/day fenfluramine as a free base or pharmaceutically acceptable salt (for example, 30 mg/day fenfluramine hydrochloride), with a daily dose of less than about 0.8 mg/kg/day, 0.7 mg/kg/day, 0.6 mg/kg/day, 0.5 mg/kg/day, about 0.4 mg/kg/day, about 0.3 mg/kg/day, about 0.25 mg/kg/day or about 0.2 mg/kg/day to about 0.1 mg/kg/day, about 0.05 mg/kg/day, or about 0.01 mg/kg/day is employed. Put differently, a preferred dose is not more than about 30 mg/day, and less than about 1 to about 0.01 mg/kg/day. Such a dose is less than the daily dose of fenfluramine suggested for administration to achieve weight loss.

The fenfluramine active agent may be administered as a suitable formulation that includes the fenfluramine active agent in a pharmaceutically acceptable vehicle with a ketogenic diet. In some aspects, the method may include administering the fenfluramine active agent at a concentration ranging from 1 mg/mL to 5 mg/mL of fenfluramine present either as a free base or pharmaceutically acceptable salt or conjugate and providing that to the patient over a period of days, weeks or months on a once a day, twice a day, three times a day or four times a day basis wherein the dose is provided to the patient at a level of 0.2 mg/kg/day or 0.7 mg/kg/day up to a maximum of 26 mg per day fenfluramine either as a free base or in a pharmaceutically acceptable salt or conjugate. The dosing is preferably provided at twelve hour intervals twice a day whereby an aspect of the invention is to reduce convulsive seizure frequency by 50% or more, 60% or more, 70% or more, 80% or more, 90% or more, 95% or more, or completely eliminate seizures in the patient over a period of 10 days, 20 days, 30 days, 50 days, 100 days or more.

The subject may be on a ketogenic diet. By “on a ketogenic diet” is meant that the patient consumes nutrition in the form of ketogenic meals, such as ketogenic breakfasts, lunches and dinners. The ketogenic diet, comprised mainly of lipid, has been used for the treatment of epilepsy in children, particularly myoclonic and akinetic seizures (Wilder, R. M. Effect of ketonuria on the course of epilepsy. Mayo Clin Bull 2: 307-ff, 1921.), and has proven effective in cases refractory to usual pharmacological means (Freeman, J. M., E. P. G. Vining. Intractable epilepsy. Epilepsia 33: 1132-1136, 1992.). Either oral or parenteral administration of free fatty acids or triglycerides can increase blood ketones, provided carbohydrate and insulin are low to prevent re-esterification in adipose tissue. Rats fed diets comprised of 70% corn oil, 20% casein hydrolysate, 5% cellulose, 5% McCollums salt mixture, develop blood ketones of about 2 MM. Substitution of lard for corn oil raises blood ketones to almost 5 mM (Veech, unpublished).

An example of a traditional 1500/day calorie ketogenic diet recommended by the Marriott Corp. Health Care Services, Pediatric Diet Manual, Revised August 1987 as suitable for a 4-6 year old epileptic child contained from 3:1 to 4:1 g of fat for each g of combined carbohydrate and protein. At each of 3 meals of the ketogenic diet the patient must eat 48 to 50 g fat, only 6 g protein and 10 to 6.5 g carbohydrate. In practice this means that at each meal the child must eat 32 g of margarine per day (about ¼ stick) and drink 92 g of heavy cream (about 100 ml), comprised mainly as medium chain length triglycerides. The diet forces the body to metabolize fats instead of carbohydrates for energy, thereby elevating the level of acetoacetate and D-3-hydroxybutyrate in the blood. These compounds are referred to as “ketone bodies,” thus the term “ketogenic” is used to describe the diet.

While the exact mechanism of action of the ketogenic diet is not well understood, it is believed that the elevated blood levels of ketone bodies have sedative effects that help to prevent seizures. In order to be effective for this purpose, however, the patient must strictly observe the diet. Vitamin and mineral supplements are included in the diet to make it nutritionally complete, since the diet is very high in fat, low in proteins, and requires the near elimination of carbohydrates. Each patient's diet is mathematically calculated based on the age, size, and activity level of the patient. Patients normally follow the diet for one to two years, at which time the patient is slowly weaned onto a normal diet. The diet has been found to be particularly effective with epileptic children. Major drawbacks are that the diet is not very palatable and that patient compliance demands complete commitment on the part of the patient and his or her family. Moreover, the diet's high fat content can increase the risk of vascular diseases, such as atherosclerosis.

In the present invention, the effective dose of a compound may be administered alone or in combination with a non-pharmacological therapy to a patient with Doose syndrome. Combination therapeutic methods are methods where a formulation having an effective dose of a compound may be used in combination with an additional therapy. As used herein, a dose of an agent, e.g., fenfluramine, refers to a therapeutically effective dose of the subject formulation containing the agent. The terms “agent,” “compound,” and “drug” are used interchangeably herein. In one embodiment, a fenfluramine formulation having an effective amount of active agent can be administered alone or in conjunction with a low carbohydrate diet, such as a ketogenic diet. As used herein, an “effective amount” is an amount of a subject compound that, when administered to an individual in one or more doses, in monotherapy or in combination therapy, is effective to reduce the occurrence of seizures by about 20%, at least about 30%, at least about 40%, at least about 50%, at least about 60%, at least about 70%, at least about 80%, or at least about 90%. In some embodiments, the subject method further includes coadministering concomitantly with the ketogenic diet a dose of fenfluramine. In some instances, the method includes administering the compound to a subject, e.g., a patient, on a ketogenic diet. In some embodiments, the method further includes administering a ketogenic diet to a patient.

The terms “co-administration” and “in combination with” include the administration of two or more therapeutic agents or therapies either simultaneously, concurrently or sequentially within no specific time limits. In one embodiment, a therapeutic agent, e.g., an amount of fenfluramine, is present in the subject's body at the same time or exerts a biological or therapeutic effect at the same time as another therapy, e.g., a ketogenic diet. In one embodiment, the therapeutic agent, e.g., an effective dose of fenfluramine, and non-pharmacological therapy, e.g., a ketogenic diet, are administered at the same time. The effective dose of the formulation of fenfluramine may be administered at the same time with a meal of the ketogenic diet. In other embodiments, the therapeutic agent and non-pharmacological therapy are administered at different times. The effective dose of the fenfluramine formulation may be administered, e.g., before or after a meal of the ketogenic diet. In certain embodiments, a first therapeutic agent or a therapy can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent or therapy.

“Concomitant administration” of a therapeutic drug or non-pharmacological therapy means administration of the compound and additional therapy (referred to as adjunctive therapy) at such time that both the drug and the non-pharmacological therapy of the present invention will have a therapeutic effect. Such concomitant administration may involve concurrent (i.e. at the same time), prior, or subsequent administration of the drug with respect to the administration of a non-pharmacological therapy. Routes of administration of the compound may vary, where representative routes of administration are described below. A person of ordinary skill in the art would have no difficulty determining the appropriate timing, sequence and dosages of administration for particular drugs or therapies of the present disclosure.

In some embodiments, a subject compound, e.g., fenfluramine, and at least one additional compound or therapy, e.g., a meal of a ketogenic diet, are administered to the subject within twenty-four hours of each other, such as within 12 hours of each other, within 6 hours of each other, within 3 hours of each other, or within 1 hour of each other. In certain embodiments, the compound and therapy are administered within 1 hour of each other. In certain embodiments, the compound and therapy are administered substantially simultaneously. By administered substantially simultaneously is meant that the compound and therapy are administered to the subject within about 10 minutes or less of each other, such as 5 minutes or less, or 1 minute or less of each other.

A method of the present invention can be practiced on any suitable subject. A subject of the present invention may be a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1.-15. (canceled)

16. A method of treating, preventing and/or ameliorating symptoms of Doose syndrome in a patient diagnosed with Doose syndrome, comprising:
administering an effective dose of fenfluramine or a pharmaceutically acceptable salt thereof to the patient.

17. The method of claim 16, wherein the patient exhibits one or more mutations in one or more of a gene selected CHD2 (15q26), GABRG2 (5q34), SCN1A (2q24.3), SCN1B (19q13.12), SLC2A1 (1p34.2), and SLC6A1(3p25.3).

18. The method of claim 17, wherein fenfluramine is adjunctively administered with an additional pharmaceutically active drug.

19. The method of claim 17, wherein the fenfluramine is the only pharmaceutically active drug administered to the patient for treating symptoms of Doose syndrome.

20. The method of claim 17, wherein the symptoms are seizures.

21. The method of claim 16, wherein the fenfluramine is administered in an amount of from 10.0 mg/kg/day to 0.01 mg/kg/day and wherein the fenfluramine is administered a dosage form selected from the group consisting of oral, injectable, transdermal, inhaled, nasal, rectal, vaginal or parenteral delivery; or wherein the fenfluramine is administered in an amount of from 0.8 mg/kg/day to 0.01 mg/kg/day.

22. The method of claim 16, wherein the fenfluramine is in an oral solution in an amount selected from the group consisting of 120 mg or less, 60 mg or less, and 30 mg or less.

23. The method as claimed in claim 16 wherein the dosage form consists essentially only of fenfluramine as the active ingredient.

24. The method as claimed in claim 16, further comprising:
administering a co-therapeutic agent selected from the group consisting of valproic acid, lamotrigine, levetiracetam, topiramate, zonisamide, rufinamide, clobazam, felbamate, ethosuximide, nitrazepam, adrenocorticotrophic hormone, methylprednisolone, prednisone, dexamethasone, clonazepam, clorazepate, perampanel, stiripentol, cannabidiol, and tetrahydrocannabinol, and pharmaceutically acceptable salts and bases thereof.

25. A method for treating a patient, comprising:
determining that a subject exhibits a mutation in a gene selected CHD2 (15q26), GABRG2 (5q34), SCN1A (2q24.3), SCN1B (19q13.12), SLC2A1 (1p34.2), and SLC6A1(3p25.3); and
administering a therapeutically effective amount of fenfluramine or a pharmaceutically acceptable salt thereof to the subject and thereby preventing and/or ameliorating seizures in the subject.

26. A method of stimulating 5-HT receptors in a patient diagnosed with Doose syndrome, comprising:
administering an effective dose of fenfluramine or a pharmaceutically acceptable salt thereof to the patient.

27. A kit for treating a symptom of Doose syndrome in a patient diagnosed with Doose syndrome, comprising:
a container comprising a plurality of doses of a formulation comprising a pharmaceutically acceptable carrier and an active ingredient comprising fenfluramine;
instructions for treating the patient diagnosed with Doose syndrome by withdrawing the formulation from the container, and administering the formulation to the patient.

28. The kit as claimed in claim 27, wherein:
the formulation is an oral solution comprising 2.5 milligram of fenfluramine in each milliliter of liquid solution; and
the instructions indicate dosing the patient based on patient weight and volume of oral solution administered.

29. The kit as claimed in claim 27, wherein the formulation is a solid oral formulation selected from the group consisting of: a tablet, a disintegrating table, a capsule, a lozenge, and a sachet wherein the fenfluramine is present in the formulation in an amount of from 5 mg to 120 mg.

30. The kit as claimed in claim 27, wherein said formulation is provided in a transdermal patch.

31. The kit of claim 27, wherein the formulation is a liquid formulation for oral administration.

32. The kit of claim 27, wherein the formulation consists essentially only of fenfluramine as the sole pharmaceutically active ingredient.

33. The method of claim 16, wherein the administering is to the subject in combination with a ketogenic diet.

34. The method as claimed in claim 33, wherein the effective dose is administered before a meal of the ketogenic diet.

35. The method as claimed in claim 34, wherein the effective dose is administered after a meal of the ketogenic diet.
